(19) United States
(12) Patent Application Publication
(10) Pub. No.: US 2023/0000411 A1
(43) Pub. Date: Jan. 5, 2023

(54) Team Wellness Tracking Devices, Systems, and Methods

(71) Applicants: Jeffrey Eyet, Lafayette, CA (US); Sara Colombo, Eindhoven (NL); Kacper Pietrzykowski, Siemianowice Slaskie (PL)

(72) Inventors: Jeffrey Eyet, Lafayette, CA (US); Sara Colombo, Eindhoven (NL); Kacper Pietrzykowski, Siemianowice Slaskie (PL)

(21) Appl. No.: 17/750,977

(22) Filed: May 23, 2022

Related U.S. Application Data
(60) Provisional application No. 63/190,468, filed on May 19, 2021.

(51) Int. Cl. A61B 5/16; G16H 40/67; A61B 5/00

(52) U.S. Cl. CPC A61B 5/165; G16H 40/67; A61B 5/002; A61B 5/742; A61B 5/6801; A61B 5/7475

(57) ABSTRACT

Wellness capture devices, systems for tracking wellness of an individual and/or a team, and methods of tracking wellness of an individual and/or a team are described. A wellness tracking device includes a housing and a cover that cooperatively define a chamber, a circuit board disposed in the chamber and including a system on a chip module, a vibration motor, a light diffuser, a first attachment member attached to the housing and a second attachment member disposed on an outer surface of the housing opposite the cover.

### FIELD

The disclosure relates to the field of wellness tracking. More particularly, the disclosure relates to wellness tracking for individuals and teams. Specific examples described herein relate to wellness capture devices, systems for tracking wellness of individuals and/or teams, and methods of tracking wellness of individuals and/or teams.

### BACKGROUND

Monitoring and tracking the mental health of members of teams can be used to improve overall team performance. Wellness, a general indictor of positive and negative mental (or emotional) states, can be monitored using relatively simple devices that permit team members to self-report their feelings to a centralized datastore. Unfortunately, existing devices, systems, and methods for tracking wellness of team members are not well adapted for the unique needs of particular teams, such as frontline health care teams. Frontline healthcare workers do not have sufficient free time to track or report their emotional states through existing methods such as surveys or mobile apps. Managers of these teams, such as nursing shift supervisors, do not have sufficient time to process complex reports of data collected from existing self-reporting devices to assess the wellness of individual team members and/or the overall wellness of the team. The demands of these roles require wellness capture devices, and related systems and methods that enable real-time, immediate assessment of the wellness of individual team members and the entire team while also enabling in-depth review and analysis of wellness data. Existing devices, systems, and methods for tracking wellness of team members do not meet these demands. Indeed, existing devices, systems, and methods for tracking wellness do not adequately address wellness tracking needs for individuals, either.

A need exists, therefore, for improved wellness capture devices, systems for tracking wellness of individuals and/or teams, and methods of tracking wellness of individuals and/or teams.

### BRIEF SUMMARY OF SELECTED EXAMPLES

Various example wellness capture devices are described.

An example wellness capture device comprises a housing and a cover that cooperatively define a chamber, a main circuit board with a system on a chip, one or more mechanical connectors, such as a USB port, one or more wireless connection antennae, such as a Bluetooth Low Energy (BLE) antenna or a Near Field Communication (NFC) antenna, one or more physical inputs, such as a tactile button configured to register manipulation of the cover, and one or more arrays of light emitting diodes (LEDs) adapted to emit a pattern of light based on an input received by a user as well as aimed at displaying the average emotional state of the individual and/or the team. A light diffuser can be included to distribute light emitted by one or more of the LEDs across a surface, such as the cover.

The portable and wearable nature of the wellness capture devices enable the input and capturing of feelings from anywhere in real time. Furthermore, the physicality of a wearable button provides convenience beyond other capture tools, such as software applications that run on mobile computing devices, which is expected to increase usage and adoption. Various systems for tracking wellness of a team are also described.

An example system for tracking wellness of a team comprises a plurality of wellness capture devices, a computing device with a software application stored in memory, the software application adapted to communicate with each wellness capture device of the plurality of wellness capture devices through a direct physical connection between the computing device and the wellness capture device or via a wireless connection between the computing device and the wellness capture device.

Various methods of tracking wellness of a team are described herein.

An example method of tracking wellness of a team comprises providing a first wellness capture device to a first member of the team; providing a second wellness capture device to a second member of the team; and operating a software application on a computing device such that the software application communicates with cloud-based computing resources to obtain data relating to the status of the first and second wellness capture devices, processes the data, and presents a visual display of the data or derivatives of the data that relate to the wellness of the first member of the team, the second member of the team, and/or the overall wellness of the team. The data is also sent back from the software application to each connected capture device, to display the average emotional state of the first member of the team, the second member of the team, and/or the overall wellness of the team directly on the capture device. Machine learning and/or artificial intelligence systems and/or processes can be included to predict a future wellness state of an individual and/or of an overall team.

Additional understanding of the inventive wellness capture devices, systems for tracking wellness of individual and/or teams, and methods of tracking wellness of individuals and/or teams can be obtained with review of the detailed description of selected examples, below, with reference to the appended drawings.

### DESCRIPTION OF FIGURES

FIG. 1 is perspective view of an example wellness capture device.

FIG. 2 is an exploded view of the example wellness capture device illustrated in FIG. 1

FIG. 3 is a side view of the example wellness capture device illustrated in FIG. 1.

FIG. 4 is a top view of the example wellness capture device illustrated in FIG. 1.

FIG. 5 is a bottom view of the example wellness capture device illustrated in FIG. 1.

FIG. 6 is an end view of the example wellness capture device illustrated in FIG. 1.

FIG. 7 is a sectional view of the example wellness capture device illustrated in FIG. 1, taken along line 7-7 in FIG. 3.

FIG. 8 is a perspective view of the example wellness capture device illustrated in FIG. 1, with the cover removed.

FIG. 9 is a schematic illustration of a system for tracking wellness of a team.

FIG. 10 is a flowchart illustration of a method of tracking wellness of a team.

### DESCRIPTION OF SELECTED EXAMPLES

The following detailed description and the appended drawings describe and illustrate various example wellness capture devices, systems for tracking wellness of teams, and methods of tracking wellness of teams. The description and illustration of these examples enable one skilled in the art to make and use examples of the inventive devices and systems, and to perform the inventive methods. They do not limit the scope of the claims in any manner.

As used herein, the term “wellness,” and related grammatical terms, refers to the emotional and/or mental wellness of a human being. The term can include an optional physical wellness component, but does not refer solely to physical wellness.

Example wellness capture devices include a housing and a cover that cooperatively define a chamber, a main circuit board with a system on a chip, one or more mechanical connectors, such as a USB port, one or more wireless connection antennae, such as a Bluetooth Low Energy (BLE) antenna or a Near Field Communication (NFC) antenna, one or more physical inputs, such as a tactile button configured to register manipulation of the cover, and one or more arrays of light emitting diodes (LEDs) adapted to emit a pattern of light based on an input received by a user as well as aimed at displaying the average emotional state of the individual and/or the overall team. A light diffuser can be included to distribute light emitted by one or more of the LEDs across a surface, such as the cover.

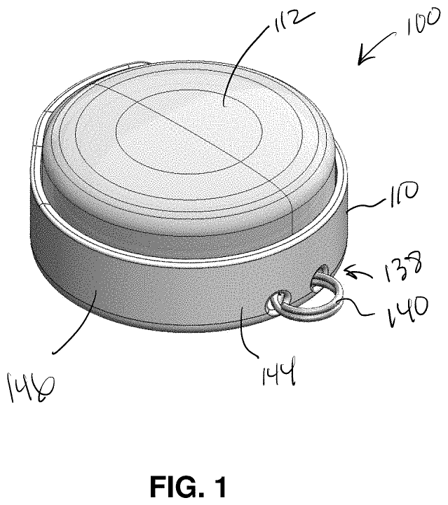

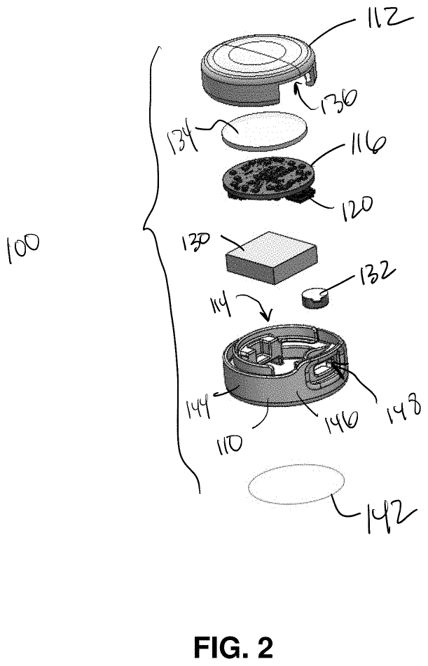

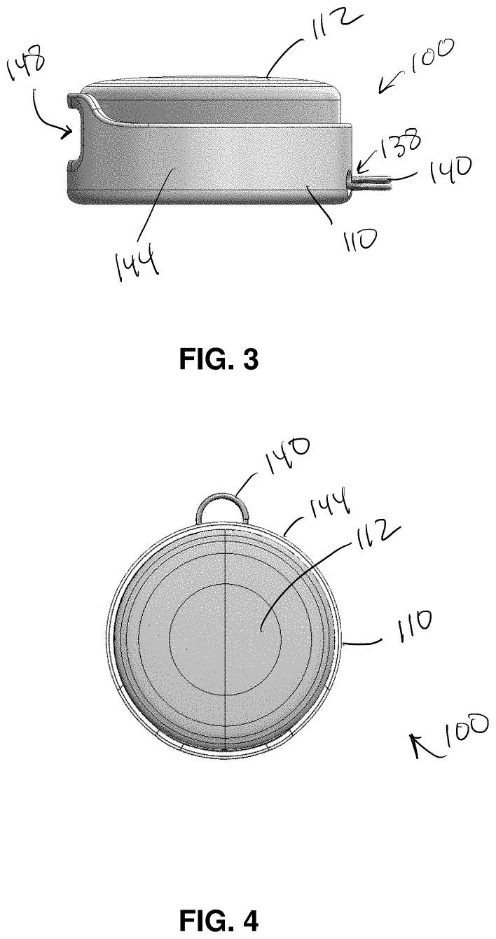

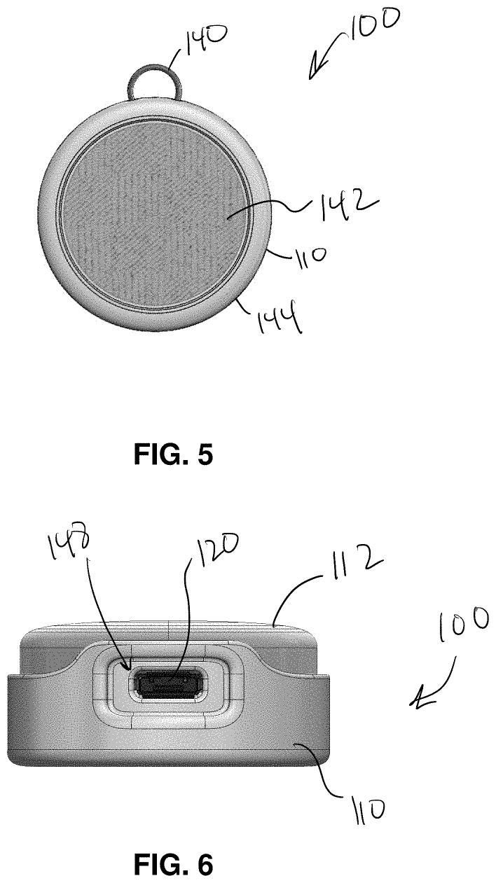

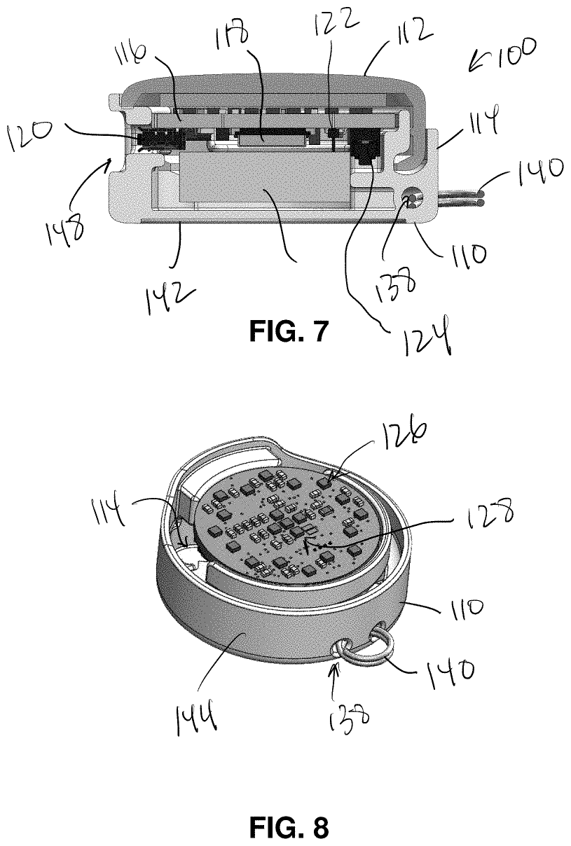

FIGS. 1, 2, 3, 4, 5, 6, 7 and 8 illustrate an example wellness capture device 100. The wellness capture device 100 comprises a housing 110 and a cover 112 that cooperatively define a chamber 114. A main circuit board 116 is disposed in the chamber 114 and includes a system on a chip module 118, a hardware connector 120, a wireless antennae 122, a tactile input button 124, a first array of LEDs 126, and a second array of LEDs 128. A battery 130 is disposed in the chamber 114 and is operably connected to the main circuit board 116 to power the wellness capture device 100. A vibration motor 132 is disposed in the chamber 114 and operably connected to the main circuit board 116 and adapted to vibrate the housing 110 when energized. A light diffuser 134 is disposed between the first 126 and second 128 arrays of LEDs and the inner surface 136 of the cover 112. The housing 110 defines a passageway 138 through which a loop 140 is passed, providing an attachment member for the wellness capture device 100. An attachment member 142 is disposed on an outer surface 144 of the housing 110, opposite the cover 112, and enables temporary securement of the wellness capture device to another surface, such as a desk, an article of clothing, or the like.

In this example, the housing 110 comprises plastic and is configured generally as a puck-shaped member, defining various contours, ridges, slots, and other features for receiving various other components of the wellness capture device 100. An outer surface 144 of the housing 110 defines a recess for receiving the attachment member 142. A circumferential outer wall 146 defines a passageway 148 that provides access to hardware connector 120. The housing 110, itself, is largely open to chamber 114, which is only fully defined in cooperation with cover 112. This configuration, in conjunction with the preferred soft and flexible nature of the cover 112, as described below, is considered advantageous at least because it provides ready access to the main circuit board 116 and its various components, the battery 130, and other internal components of the wellness capture device 100.

Passageway 138 provides a structure through which another member, such loop 140, can be passed to facilitate wearing of the wellness capture device around the wrist, neck, or other body portion. Also, passageway 138 and loop 140 provide structure for securement to other personal items for easy transport, storage, and wearing, such as lanyards, keychains, and the like.

In this example, the cover 112 comprises a relatively soft and relatively flexible material as compared to the material of the housing 110. Furthermore, and critically, the cover 112 is translucent, allowing light emitting by one or more of the LEDs from one or both of the first 126 and second 128 arrays of LEDs to pass through the cover 112. Translucency is considered advantageous to the overall functioning of the wellness capture device as a team wellness tracking tool as this property facilitates use of the capture device 100 in related systems and methods. Translucency is optional, though, and alternative materials or systems can be used, including any materials or systems that allow the surface to be aesthetically pleasing while enabling the provision of visual feedback. Also, the relatively soft and relatively flexible nature of the material of the cover 112 is considered advantageous at least because it provides a desirable human interface while also providing a suitable barrier to entry to internal components of the wellness capture device 100, as described above, its suitability for use in human-machine interface components, and its aesthetically pleasing nature. Examples of suitable materials for the cover 112 include, but are not limited to, flexible plastics, elastomeric materials, rubbers, silicone, and thermoplastic polyurethane.

The main circuit board 116 can comprise any suitable circuit board providing the desired system on a chip, connectivity, input, and LED control. While the illustrated example includes a hardware connector 120, a wireless antenna 122, a tactile input button 124, a first array of LEDs 126, and a second array of LEDs 128, any suitable number and type of connectivity, input, and light and/or color emission elements can be included. In this embodiment, hardware connector 120 comprises a USB-A port, wireless antenna 122 comprises a Bluetooth Low Energy antenna, and the tactile input button 124 comprises a push button microswitch. In addition to the input button 124, other inputs can be included, if desired, such as a microphone, sensors, such as an accelerometer, for tracking a user's temperature, number of steps taken, and other parameters that can go to the user's overall wellness. In one embodiment, a microphone is embedded in tactile input button 124 such that pressing the input button 124 activates the microphone for sound input and recording and/or forwarding of the input received by the microphone.

The system on a chip (SoC) 118 module can comprise any suitable SoC that provides the desired ability to receive input from the tactile input button, connectivity through the hardware connector 120 and wireless antenna 122, and ability to control the first 126 and second 128 arrays of LEDs. Examples of suitable SoC modules include, but are not limited to, the nRF52832 general purpose, multi-protocol SoC module available from Nordic Semiconductor.

Each of the first 126 and second 128 array of LEDs comprise a plurality of LEDs arrange on the main circuit board 116. The first array of LEDs 116 is disposed near the center of the main circuit board 116 and the second array of LEDs 118 is disposed near the periphery of the main circuit board 116, radially outward from the first array of LEDs 116. This structural arrangement is considered advantageous at least because it provides a desired ability to display different light patterns meaningful to wellness tracking (e.g., inner LEDs on, outer LEDs off; inner LEDs off, outer LEDs on; inner LEDs on, outer LEDs on, etc.). Color LEDs can be used, but are not required. If color LEDs are required, LEDs that emit light of any colors can be used.

The battery 130 can comprise any suitable battery, including a conventional calculator battery, a watch battery, a rechargeable battery, or 9-volt battery, a AAA battery, or any combination of batteries. The battery selected for a wellness capture device according to a particular embodiment need only be able to power the wellness capture device for a desirable period of time.

The light diffuser 134 is a section of translucent material, such as plastic, the diffused light emitted by one or more of the LEDs of the first 126 and second 128 arrays of LEDs, effectively diffusing the light before it passes through the cover 112. This ensures that the light being emitted by the LED or LEDs is spread across a relatively large portion of the outer surface 150 of the cover 112.

The attachment member 142 is a section of material adapted to temporarily secure the wellness capture device to another surface, either alone or in cooperation with structural features of the other surface. For example, the attachment member 142 can comprise an adhesive layer that can adhere the wellness capture device to another surface, such as an article of clothing, personal bag, etc. Alternatively, the attachment member can comprise one portion of the hook and loop attachment pairing, which enables formation of a temporary securement between the wellness capture device and another surface having a mating portion of the hook and loop attachment pairing, such as an article of clothing or the like.

An app for a mobile computer, such as a mobile phone, is also contemplated. The app provides the same input and capture capabilities as the wellness capture devices, but eliminates the need for a separate device. If used in addition to the separate device, the app can be employed to input additional details about the user's emotional state, as well as to access more information about the individual's and the team's wellness.

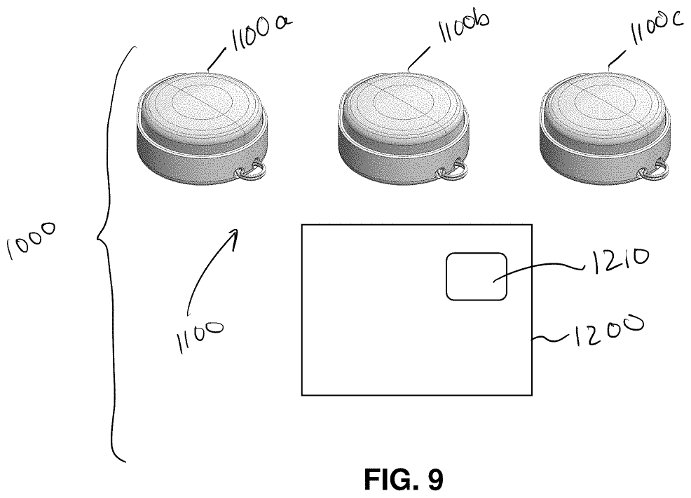

FIG. 9 is a schematic representation of a system 1000 for tracking wellness of a team. The system comprises a plurality 1100 of wellness capture devices, a computing device 1200 with a software application 1210 stored in memory, the software application 1210 adapted to communicate with cloud-based computing resources to retrieve data relating to inputs received by each wellness capture device 1100a, 1100b, 1100c of the plurality of wellness capture devices 1100. The software application 1210 may also be adapted to communicate with each wellness capture device 1100a, 1100b, 1100c of the plurality of wellness capture devices 1100 through a direct physical connection between the computing device 1200 and the wellness capture device 1100a, 1100b, 1100c or via a wireless connection between the computing device 1200 and the wellness capture device 1100a, 1100b, 1100c.

Each wellness capture device 1100a, 1100b, 1100c comprises a wellness capture device according to an embodiment of the invention, and can be a wellness capture device described and illustrated herein. Also, while the illustrated system 1000 includes three wellness capture device 1100a, 1100b, 1100c, any suitable number of wellness capture devices can be included. The number of wellness capture devices included in a system for tracking wellness of a team according to a particular embodiment will depend on the number of members of the team for which wellness is to be tracked.

The computing device 1200 can comprise any suitable computing device having memory for storing the software application, a display for presenting information related to the plurality 1100 of wellness capture devices, and any desired input and/or user interface elements, such as buttons, touch screen capabilities, microphones, etc. The computing device 1200 has conventional networking capabilities, such as a wired networking port, such as an Ethernet port, and/or a wireless radio, such as a WiFi capable radio. Examples of suitable computing devices include a desktop computer, a portable computer, such as a laptop, a mobile telephone, such as a smartphone or other mobile telephone adapted to store and execute software applications, and a tablet computer.

The software application 1210 can comprise any suitable application that can be stored on memory of the computing device 1200 and executed by the computing device 1200 such that the software application 1210 communicates with cloud-based computing resources, such as databases, servers, APIs, serverless scripts, etc. to retrieve data relating to the inputs received by each wellness capture device 1100a, 1100b, 1100c of the plurality 1100 of wellness capture devices. Preferably, the software application 1210 is adapted to process the data and present a visual display of the data or derivatives of the data that relate to the wellness of individual team members to which the individual wellness capture devices 1100a, 1100b, 1100c have been assigned and/or the overall wellness of the team.

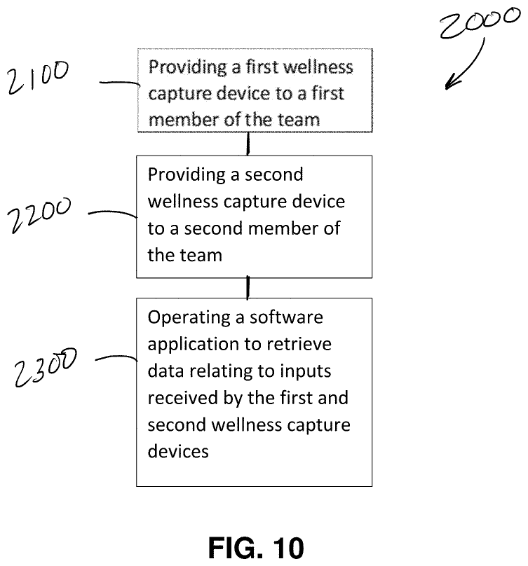

FIG. 10 is a flowchart representation of an example method 2000 of tracking wellness of a team. A first step 2100 comprises providing a first wellness capture device to a first member of the team. Another step 2200 comprises providing a second wellness capture device to a second member of the team. Another step 2300 comprises operating a software application on a computing device such that the software application communicates with cloud-based computing resources to retrieve data relating to inputs received by the first and second wellness capture devices, processes the data, and presents a visual display of the data or derivatives of the data that relate to the wellness of the first member of the team, the second member of the team, and/or the overall wellness of the team. The software application also transmits the data that relate to the average wellness of the first member of the team, the second member of the team, and/or the average overall wellness of the team to each connected capture device, which displays such data directly to the user of the respective capture device.

Each wellness capture device comprises a wellness capture device according to an embodiment of the invention, and can be a wellness capture device described and illustrated herein. Also, while the illustrated method 2000 includes two wellness capture devices and two team members, any suitable number of wellness capture devices can be included. The number of wellness capture devices used in a method of tracking wellness of a team according to a particular embodiment will depend on the number of members of the team for which wellness is to be tracked. A suitable number of additional steps of providing a wellness capture device to a member of the team can be included to scale the method to any desired team size. Additionally, step 2300 can be adjusted so that operation of the software application on the computing device is performed in a manner such that the software application communicates with the total number of wellness capture devices included in the method.

Any suitable number of wellness capture devices can be used in a method according to a particular embodiment. The number of wellness capture devices included in a method of tracking wellness of a team according to a particular embodiment will depend on the number of members of the team for which wellness is to be tracked. Steps 2100 and 2200 are performed by assigning an individual wellness capture device to an individual member of a team such that each individual member can operate the assigned wellness tracking device to provide input to the wellness tracking device relating to the wellness of the individual.

Step 2300 is performed by running a software application that is adapted to communicate with cloud-based computing resources to retrieve data relating to inputs received by the first and second wellness capture devices. For example, a software application can retrieve data stored in a remote database, the data representing inputs received by the individual wellness capture devices assigned to individual team members in steps 2100 and 2200.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

I claim:

1. A wellness tracking device, comprising
a housing and a cover that cooperatively define a chamber;
a main circuit board disposed in the chamber and including a system on a chip module, a hardware connector, a wireless antennae, a tactile input button, a first array of LEDs, and a second array of LEDs;
a battery disposed in the chamber and operably connected to the main circuit board;
a vibration motor disposed in the chamber and operably connected to the main circuit board and adapted to vibrate the housing when energized;
a light diffuser disposed between the first and second arrays of LEDs and an inner surface of the cover;
a first attachment member extending through a passageway defined by the housing; and
a second attachment member disposed on an outer surface of the housing opposite the cover, the second attachment member adapted to enable temporary securement of said wellness capture device to a surface.

2. A system for tracking wellness of a team, said system comprising:
a plurality of wellness capture devices according to claim 1; and
a computing device with a software application stored in memory, the software application adapted to communicate with cloud-based computing resources to retrieve data relating to inputs received by each wellness capture device of the plurality of wellness capture devices.

3. A method of tracking wellness of a team, said method comprising:
providing a first wellness capture device according to claim 1 to a first member of said team;
providing a second wellness capture device according to claim 1 to a second member of said team; and
operating a software application on a computing device such that the software application communicates with cloud-based computing resources to retrieve data relating to inputs received by the first and second wellness capture devices;
processing the data to determine the wellness of the first member of the team, the second member of the team, and/or the overall wellness of the team.
transmitting data that relate to the average wellness of the first member of the team, the second member of the team, and/or the average overall wellness of the team to the first wellness capture device and the second wellness capture device;
displaying data that relate to the average wellness of the first member of the team, the second member of the team, and/or the average overall wellness of the team on the first wellness capture device; and
displaying data that relate to the average wellness of the first member of the team, the second member of the team, and/or the average overall wellness of the team on the second wellness capture device.
